(19) United States
(12) Patent Application Publication
(10) Pub. No.: US 2019/0254915 A1
(43) Pub. Date: Aug. 22, 2019

(54) WEARABLE ASSISTIVE DEVICE HAVING IMPROVED WAIST SUPPORT

(71) Applicant: LG ELECTRONICS INC, Seoul (KR)

(72) Inventors: Bohyun NAM, Seoul (KR); Kyu Tae PARK, Seoul (KR); Jung Kyu SON, Seoul (KR); Seonil YU, Seoul (KR); Wonjun LEE, Seoul (KR)

(21) Appl. No.: 16/274,662

(22) Filed: Feb. 13, 2019

Related U.S. Application Data
(60) Provisional application No. 62/730,399, filed on Sep. 12, 2018.
(60) Provisional application No. 62/730,400, filed on Sep. 12, 2018.
(60) Provisional application No. 62/730,412, filed on Sep. 12, 2018.
(60) Provisional application No. 62/730,420, filed on Sep. 12, 2018.

(30) Foreign Application Priority Data
Feb. 20, 2018 (KR) 10-2018-0019992

(51) Int. Cl. A61H 3/00; A61F 5/01

(52) U.S. Cl. CPC A61H 3/008; A61F 5/0102; A61H 2003/007; A61H 2201/164; A61H 2201/165; A61H 2201/1676; A61H 2201/1645

(57) ABSTRACT

A wearable assistive device may include a main frame that covers a pelvis of a user, a subframe installed on a front surface of the main frame to secure a waist of the user, and a leg assembly installed at opposite ends of the main frame and formed to extend downward along each leg of the user to generate an assistive force that assists the user. The subframe may include a main waist support that supports a back of the user, a first support hingedly coupled to a first end of the main waist support to support a first side of the waist of the user, and a second support hingedly coupled to a second end of the main waist support to support a second side of the waist of the user.

### CROSS-REFERENCE TO RELATED APPLICATION

This application claims priority under 35 U.S.C. § 119 to U.S. Provisional Patent Application Nos. 62/730,399, 62/730,400, 62/730,412, and 62/730,420, all filed on Sep. 12, 2018, and also priority to Korean Application No. 10-2018-0019992, filed on Feb. 20, 2018, whose entire disclosures are herein incorporated by reference.

### BACKGROUND

### 1. Field

This application relates to assistive and/or rehabilitative technology.

### 2. Background

In assistive and/or rehabilitative technology, a wearable assistive device such as a wearable robot, e.g., exoskeleton, may assist or augment a movement of a user. The wearable assistive device may be a kind of robot having a multi-joint skeletal structure to assist a user during walking. The wearable assistive device may assist the power of the user by providing a driving force or an assistive force generated from a driving means such as a motor to the user.

JP Patent Application Publication No. 2006-087533A discloses a conventional walking assisting apparatus (i.e., a power assisting robot). FIG. 1 is a perspective view explaining a waist support of the walking assisting apparatus. FIGS. 2 to 4 are schematic views explaining a mounting or securing of the waist support of FIG. 1. For ease of explanation, some of the reference numerals disclosed in JP Patent Application Publication No. 2006-087553A may be changed.

As shown in FIG. 1, a waist support 11 of the walking assisting apparatus may be formed in a C-shape. Left and right opening/closing portions or ends 11b and 11c of a main waist support 11a may be made of an elastically deformable material, and there may be a space between the left and right ends 11b and 11c. The user may apply a force to the left and right ends 11b and 11c to elastically deform or mold the waist support 11, thereby sufficiently opening the left and right ends 11b and 11c so as to place his waist into the waist support 11.

As shown in FIGS. 2 to 4, even when a width W2 of a waist U of the user is wider than a width W1 between the left and right ends 11b and 11c, the user may don the waist support 11 on his waist U by moving the left and right ends 11b and 11c. Since a front of the waist U may be exposed in the space between the left and right ends 11b and 11c, the walking assisting apparatus may not be properly secured to the user and may be shaken or displaced.

When the user tries to take off the walking assisting apparatus, the left and right ends 11b and 11c have to be elastically deformed again. However, it may be difficult to actually take off the walking assisting apparatus since the left and right ends 11b and 11c have already opened a certain extent and may press an elastic force against the waist U of the user.

When the waist is properly secured, an assistive force of the walking assisting apparatus generated at a hip joint may help the walking assisting apparatus to function properly. Since securing the waist with the waist supporting member 11 is imperfect, there is a problem in that it may be difficult to provide a proper or efficient assistive force to the user. Furthermore, there also is a problem in that the user may be unable to walk due to the imperfectly supplied assistive force, or the user may walk in an unstable state.

Since a waist size and shape of the user may vary based on factors such as gender, size, or age, it may be difficult to flexibly accommodate various waist sizes and shapes with the waist support 11 having an open front. As a result, the waist support 11 may be applicable only to a personalized walking assisting apparatus, and it may be difficult to apply the waist support 11 to a general-purpose walking assisting apparatus.

The above reference is incorporated by reference herein where appropriate for appropriate teachings of additional or alternative details, features and/or technical background.

### BRIEF DESCRIPTION OF THE DRAWINGS

The embodiments will be described in detail with reference to the following drawings in which like reference numerals refer to like elements wherein;

FIG. 1 is a perspective view showing a waist support of a conventional walking assisting apparatus;

FIGS. 2 to 4 are schematic views of a donning process of the waist support of FIG. 1;

FIG. 5A is a perspective view of a wearable assistive device according to one embodiment;

FIG. 5B is a perspective view of a wearable assistive device according to an embodiment;

FIG. 6 is a side view of the wearable assistive device of FIG. 5A;

FIG. 7 is a front view of a subframe of FIG. 5A;

FIG. 8 is a front view of the subframe of FIG. 7 in which a portion of a configuration is omitted;

FIG. 9 is a rear view of the subframe of FIG. 8;

FIG. 10 is a side view of the subframe of FIG. 8;

FIG. 11 is a side view of the subframe opposite the side view of FIG. 10;

FIG. 12 is a cross-sectional view of the subframe of FIG. 7 taken along line E-E.

FIG. 13 is a plan view of the subframe of FIG. 7 in which a portion of a configuration is omitted;

FIG. 14 is a cross-sectional view of the subframe of FIG. 13;

FIGS. 15 and 16 are schematic views explaining a method of securing a waist of a user with the subframe of FIG. 7; and

FIGS. 17 to 19 are schematic views explaining a method of using a dial to put on a take off a waist belt.

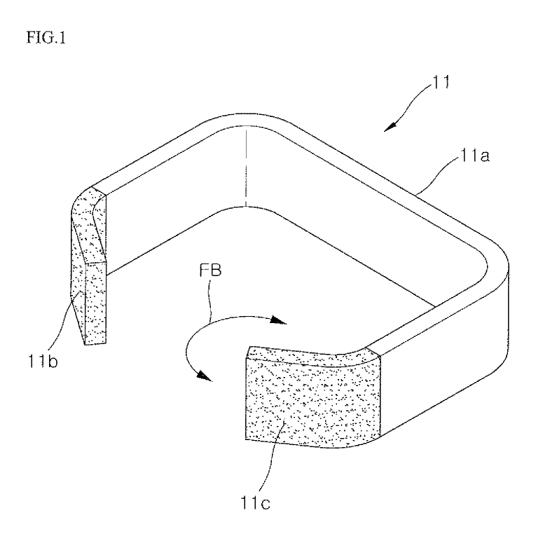

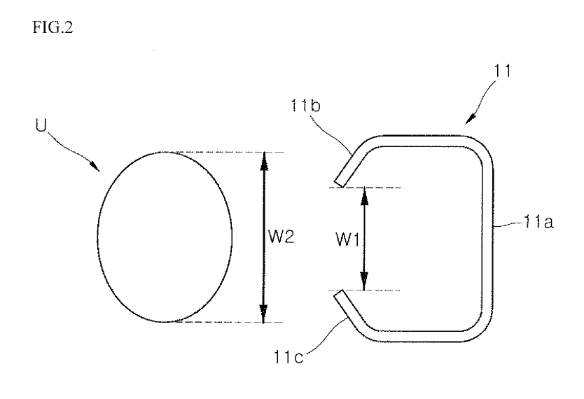

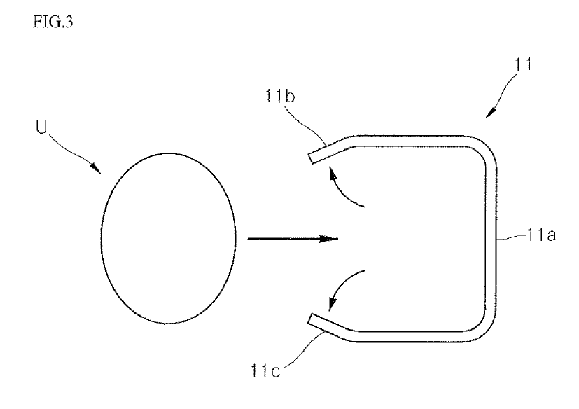

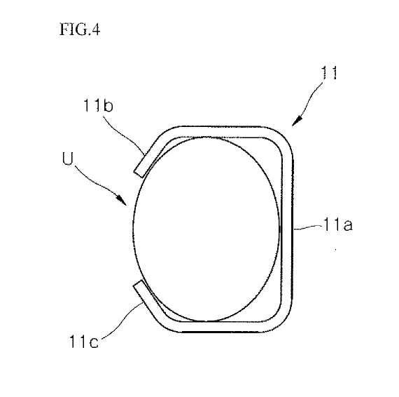

### DETAILED DESCRIPTION

Hereinafter, a structure of a wearable assistive device such as a wearable robot A, and more specifically an exoskeleton, used by a user will be briefly described as an example. However, embodiments disclosed herein are not limited to an exoskeleton that fits onto a lower body of the user. For example, an exoskeleton A that fits onto an upper body of the user may be provided. As another example, a full body exoskeleton or exoskeleton A that fits onto the upper body and the lower body of the user may also be provided.

Hereinafter, a user will be referred to as a person who wears the exoskeleton, and an assistant will be referred to as a person who assists the user in wearing the exoskeleton A or who transfers or transports the exoskeleton A.

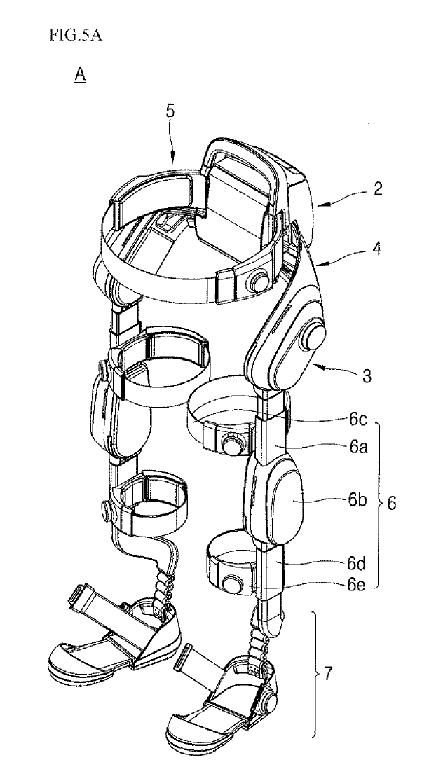

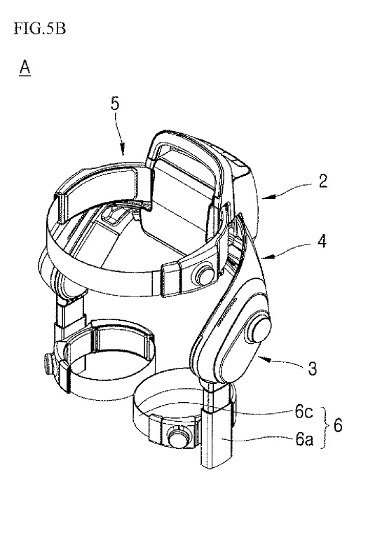

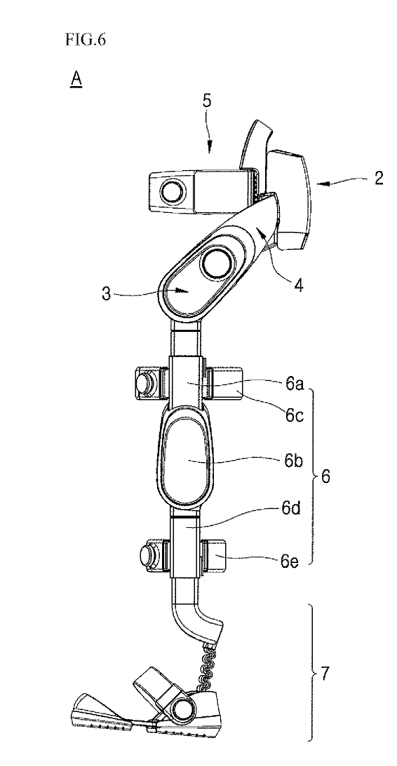

FIG. 5A is a perspective view of an exoskeleton A according to an embodiment. FIG. 5B is a perspective view of a wearable assistive device according to an embodiment. FIG. 6 is a side view of the exoskeleton A of FIG. 5A. Referring to FIGS. 5A and 6, an exoskeleton A may be worn on a lower body of the user, and may assist a lower body power or strength of the user during a movement such as walking, bending, or lifting. The exoskeleton A may include a lumbar/back frame 2, an actuated hip joint 3, a main frame 4 and a waist/pelvic frame or subframe 5 that are mounted on or secured to a pelvis or a waist of the user, a leg or leg assembly 6 that may be secured to a leg of the user, and a foot support or foot assembly 7 that may secure to and support a shoe or foot of the user. The exoskeleton A may further include a main controller housed in the lumbar/back frame 2 and a subcontroller provided at the actuated hip joint 3 to control a function or movement of the exoskeleton A.

Details of the main controller and the subcontroller can be found in U.S. application Ser. No. 16/274,584 (Attorney Docket No. DAE-0073) filed on Feb. 13, 2019 and U.S. application Ser. No. 16/274,613 (Attorney Docket No. DAE-0074) filed on Feb. 13, 2019, the entire contents of which are hereby incorporated by reference.

The lumbar/back frame 2 may be provided on the main frame 4 and positioned at a rear of the user. The main controller may adjust a width of the main frame 4 based on a body size of the user. In addition, the main controller may include a battery pack or power supply that supplies power to operate the exoskeleton A.

The subframe 5 may be coupled to a front surface of the lumbar/back frame 2, and a front surface of the main frame 4. The subframe 5 may secure to the waist of the user via a belt or waist belt to be described later, the waist belt having a length that may be adjusted via a dial. A section of the subframe 5 that is likely to contact the waist of the user may include a shock absorbent material such as a soft foam material, to improve comfort.

The main frame 4 may support the lumbar/back frame 2 and may have a shape to cover the hip or pelvis of the user. The main frame 4 may extend from a first side, e.g., left side, of the user to a second side, e.g., right side, of the user. The main frame 4 may support the waist or the pelvis of the user. The main frame 4 may have a substantial “U”-shape, and a bent or curved portion of the main frame 4 may be positioned at a rear of the user. Opposite ends or sides of the main frame 4 may extend downward along the hips or pelvis, e.g., ilium of the user. Opposite sides of the main frame 4 may therefore be inclined, and the subcontroller may be provided on these sides of the main frame 4.

The subcontroller may adjust a strength or magnitude of an assistive force that assists a power or strength of the user. A hip drive may include a driving means, e.g., a motor or actuator (hydraulic, pneumatic, or electrical), to generate an assistive force and may be provided within the actuated hip joint 3. The assistive force may be adjusted in a rotary dial manner. The actuated hip joint 3 or the main frame 4 may be provided with an indicator to indicate the magnitude of the assistive force via a lamp, light, or light emitting diode (LED), and the leg assembly 6 may be coupled to a lower side of the actuated hip joint 3.

A leg assembly 6 may be provided at an end of the main frame 4 and may be formed to extend downward along the leg of the user so as to generate an assistive force that assists the user's power or strength. There may be a pair of leg assemblies 6, each coupled to an end of the main frame 4, as the wearable apparatus A may be worn on both legs of the user. Each leg assembly 6 may include an upper leg frame 6a that may be secured to a thigh of the user via a leg belt or strap 6c, a lower leg frame 6d that may be secured to a calf or lower leg of the user via a leg belt or strap 6e, and an actuated joint provided between the upper leg frame 6a and the lower leg frame 6d to supply an assistive force. The leg belts 6c and 6e may each have a length or size adjusted in a one-touch dial manner.

The upper leg frame 6a, the lower leg frame 6d, and the actuated joint 6b may be arranged along a side of the leg of the user so as not to interfere with a bending motion, allowing a smooth bending motion of joints when the user moves (e.g., walks, bends, or lifts). The exoskeleton A may also easily bend at the actuated hip joint 3 and the actuated joint fib.

The upper leg frame 6a may rotate at the actuated hip joint 3 so that the user may move his thigh forward and backward at the hip in a sagittal plane of motion. The lower leg frame 6d may rotate at the actuated joint 6b so that the user may move his calf forward and backward at the knee in a sagittal plane of motion.

An upper portion of the upper leg frame 6a may be extended outward and inward in a frontal plane of motion by a predetermined angle by a hip joint structure of the main frame 4. Further, the upper leg frame 6a and the lower leg frame 6d may have a multi-joint structure capable of adjusting an angle or inclination of the upper and lower leg frames 6a and 6d inward or outward in the frontal plane.

The actuated joint 6b may have a knee drive, which may include a motor and a gear set or actuator (hydraulic, pneumatic, or electrical). The knee drive may generate an assistive force that assists the power or strength of the user when the user moves his leg. The knee drive may be replaced with a constituent element other than the motor and the gear set capable of generating a proper assistive force. Details of the main frame 4, the actuated hip joint 3, the actuated joint 6b, the leg assembly 6, and the hip and knee drives can be found in U.S. application Ser. No. 16/282,458 (Attorney Docket No. DAE-0075) filed on Feb. 22, 2019, the entire contents of which are incorporated herein by reference.

The foot support 7 may be coupled to a lower end of the lower leg frame 6d. The foot support 7 may be installed at a lower end of the leg assembly 6 to support the user's foot, shoe, or sock. It may be possible to adjust a length of a portion of the foot support 7 where a user's shoe may be inserted, thereby accommodating various shoe sizes. Details of the foot support 7 can be found in U.S. application Ser. No. 16/274,560 (Attorney Docket No. DAE-0072) filed on Ser. No. 16/274,798 and U.S. application Ser. No. ______ Feb. 13, 2019 (Attorney Docket No. DAE-0095) filed on Feb. 13, 2019, the entire contents of which are incorporated herein by reference.

The exoskeleton A is not limited to a lower body exoskeleton. For example, exoskeleton A may instead be an upper body exoskeleton, which may have a lumbar/back support 2 housing a main controller, a main frame 4, a subframe 5, a limb assembly 6 extending upward from the main frame 4, and at least one actuated joint 3 or 6b. An exoskeleton A may also fit onto a complete or entire body of the user to accommodate both leg and arm limbs.

Furthermore, embodiments disclosed herein may not be limited to a complete lower body exoskeleton based on an intended use of the exoskeleton. Referring to FIG. 5B, the actuated joint 6b, the lower leg frame 6d, the leg belt 6e, and the foot support 7 may be omitted. Thus, an exoskeleton A may provide assistance to the user at the actuated hip joint 3, and may include a main frame 4, a lumbar/back frame 2, and a leg assembly 6 that includes only an upper leg frame 6a that secures to a thigh of the user via a leg belt 6c. Such an upper leg exoskeleton A may have a hip structure substantially the same as or similar variations to the hip structure of the main frame 4 described in detail herein.

Since the exoskeleton A may be equipped with various constituent elements and may be made primarily of metal, it may weigh from several kilograms to tens of kilograms. Therefore, the exoskeleton A may be generally stored in a separate place, and may be transferred or transported from a storage place to the user, if necessary. The exoskeleton A according to an embodiment may be supported on an adaptive assistive and/or rehabilitative device (AARD) that may store, charge, and transport the exoskeleton A, among other functions. The AARD may serve as a walking aid for the user when the user wears the exoskeleton A, and may also serve as a chair on which the user may sit when wearing the exoskeleton A. Details of the AARD B can be found in U.S. application Ser. No. 16/274,790 (Attorney Docket No. DAE-0068) filed on Feb. 13, 2019, the contents of which are incorporated herein by reference in their entirety.

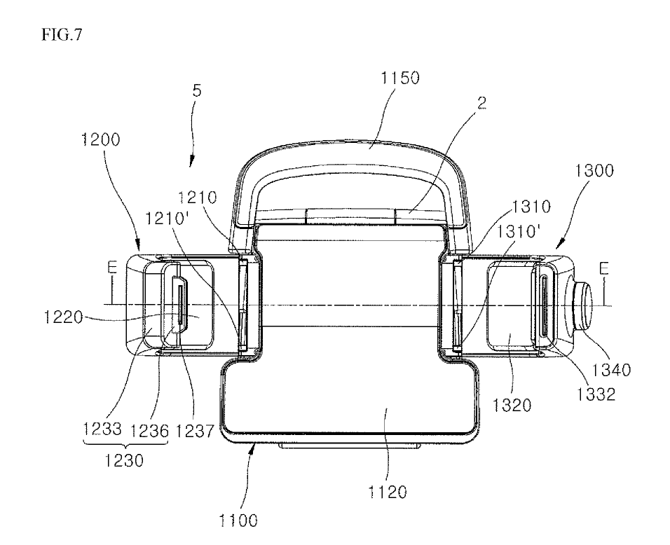

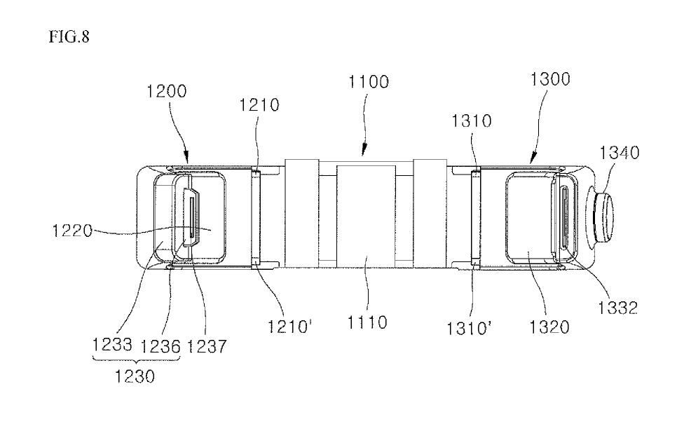

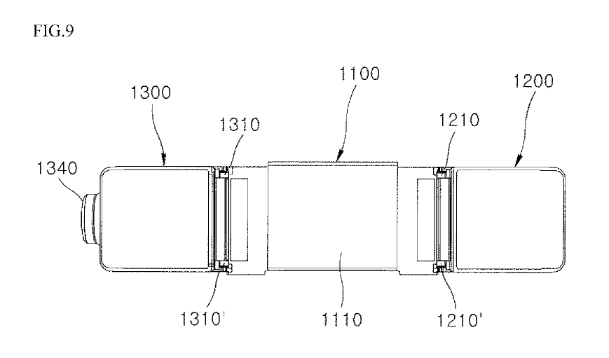

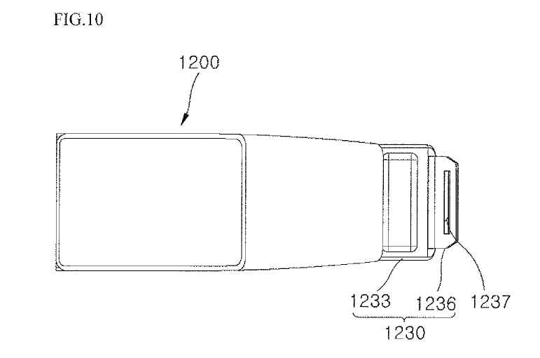

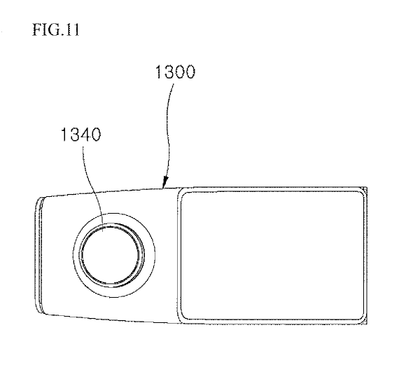

Referring to FIG. 7, a handle 1150 may be installed at an upper end of the main waist support 1100 and may be formed to be parabolic upward, or may form a general U-shape upwards or curve in an upward direction. The handle 1150 may be used when the assistant assists the user in wearing the exoskeleton A or when the exoskeleton A is transported to and from the user. The handle 1150 is omitted in FIGS. 8-11, 13, and 14 so that a structure the main waist support 1100 can be clearly shown.

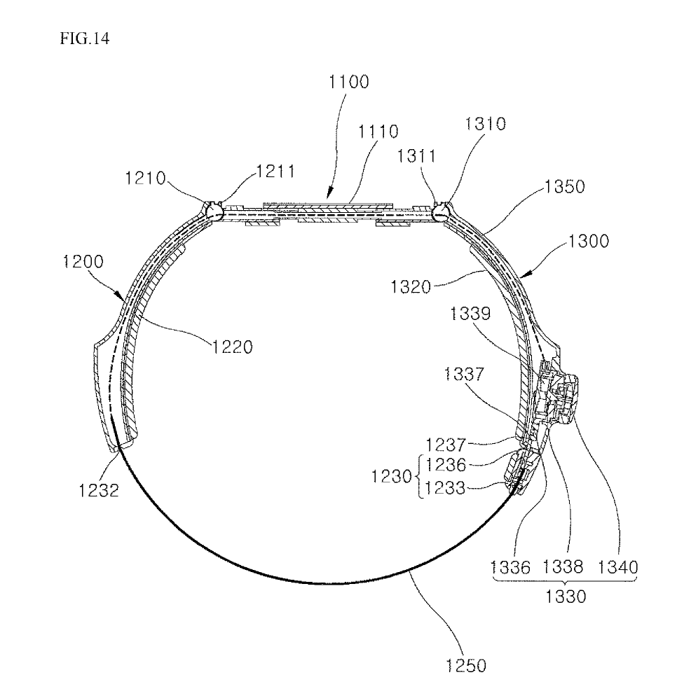

Referring to FIGS. 7 to 14, the subframe 5 may include a main waist support 1100, at least one first support or arm 1200, at least one second support or arm 1300, first hinge coupling portions or hinge brackets 1210 and 1210′, second hinge coupling portions or hinge brackets 1310 and 1310′, a handle 1150, a belt coupling structure or buckle 1230, and a wire winding structure, dispenser, or a wire winder 1330 (see FIG. 14). The wire winder 1330 may be or include a reel, spindle, or bobbin. The first and second hinge brackets 1210, 1210′, 1310, and 1310′ may also be referred to as hinge knuckles. As an alternative, the subframe 5 may omit the first support 1200 and/or the second support 1300.

The main waist support 1100 may support a back, e.g., lumbar, of the user. The main waist support 1100 may include a central support 1110 that may be hingedly coupled to each of the first and second supports 1200 and 1300, and a supporting module or inner support 1120 that may be provided on a front surface of the central support 1110 to be in direct contact with the back of the user. A front surface of the inner support 1120 may include shock absorbent material such as a soft foam or flexible polymer layer or sheet material to provide comfort and minimize impact to the user. The inner support 1120 is omitted in FIGS. 8-11, 13, and 14 so that a structure the main waist support 1100 can be clearly shown.

The central support 1110 may be formed in multiple pieces, or may be formed integrally. The central 1110 and/or the inner support 1120 may be configured to couple to the lumbar/back support 2. For example, the lumbar/back support 2 may have a plate or protrusions to be inserted into a space of the inner support 1120, but embodiments disclosed herein are not limited to such a coupling relationship. The central support 1110 may be coupled to the main frame 4. There may be an optional plate structure behind the central support 1110 used to couple the subframe 5 to the main frame 4 and/or the lumbar/back frame 2.

A first end of the central support 1110 may be provided with the first hinge brackets 1210 and 1210″ that enable hinge coupling with the first support 1200. A second end of the central support 1110 may be provided with the second hinge brackets 1310 and 1310′ that enable hinge coupling with the second support 1300.

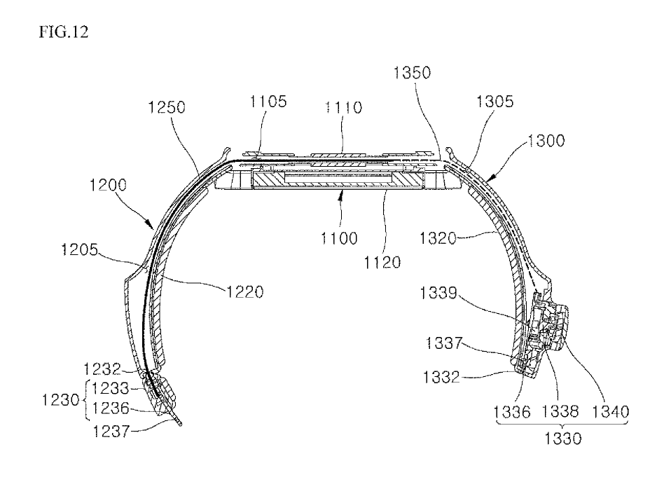

A first guide passage or first guide 1105 (FIG. 12), in which a belt 1250 and a wire 1350 are accommodated and moved may be formed within the main waist support 1100; i.e., within the central support 1110. The belt 1250 may have a first end and a second end.

The first support 1200 may be hingedly coupled to a first end of the main waist support 1100; i.e., a first end of the central support 1110, to support a first side of the waist of the user. The first support 1200 may be hingedly coupled to the first hinge brackets 1210 and 1210″ installed at the first end of the central support 1110.

The first support 1200 may be made of a duralumin alloy, a titanium alloy, or a carbon fiber material, so as to ensure durability and a light weight, but may be not limited thereto. An inner side surface of the first support 1200 may include a first impact portion or cushion 1220 made of a soft foam material or a shock-absorbent material or flexible polymer layer or sheet so as to minimize an impact applied to the first side of the waist of the user. The first impact portion 1220 may be attached to the inner side surface of the first support 1200 in a Velcro manner, for example, to facilitate cleaning and replacement. Alternatively, the first impact portion 1220 may be permanently attached to the inner side surface of the first support 1200 via a resin or glue.

A second guide passage or second guide 1205 in which the belt 1250 and the wire 1350 are accommodated and moved may be formed within the first support 1200. The buckle 1230 may be detachably installed at an end of the first support 1200. Also, a belt hole or belt opening 1232 may be formed at an end of the first support 1200 through which the belt 1250 may be withdrawn from and retracted into. The belt opening 1232 may also be referred to as a belt withdrawing and inserting hole. To withdraw the belt 1250 from the belt opening 1232, a user may pull the belt 1250 out of the second guide 1205 from the buckle 1230.

The second support 1300 may be hingedly coupled to the second end of the main waist support 1100; i.e., the second end of the central support 1110. The second support 1300 may be hingedly coupled to the second hinge brackets 1310 and 1310′ installed at the second end of the central support 1110. The second support 1300 may be made of a duralumin alloy, a titanium alloy, or a carbon fiber material, so as to ensure durability and a light weight, but may be not limited thereto.

An inner side surface of the second support 1300 may include a second impact portion or cushion 1320 made of a soft foam material or shock absorbent material or flexible polymer layer or sheet so as to minimize an impact applied to the second side of the waist of the user. The second impact portion 1320 may be attached to the inner side surface of the second support 1300 in a Velcro manner, for example, so as to facilitate cleaning and replacement. Alternatively, the second impact portion 1320 may be permanently attached to the inner side surface of the second support 1300 via a resin or glue.

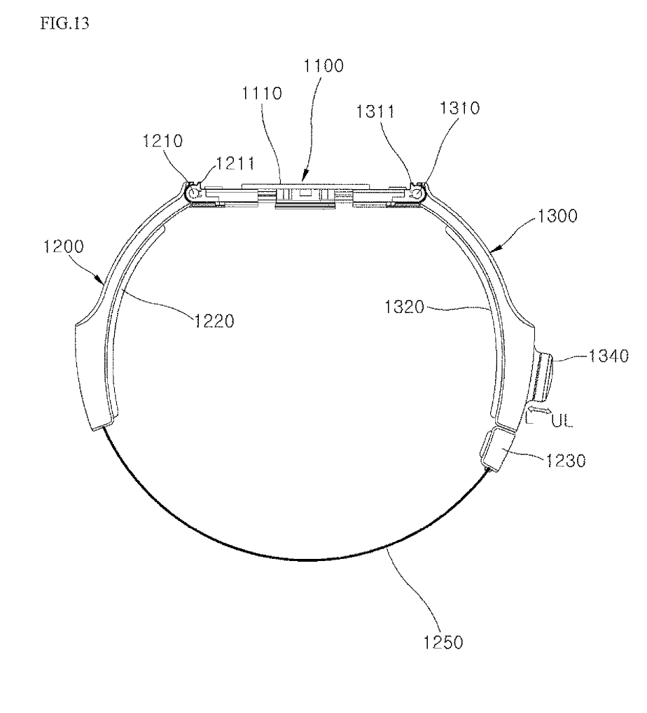

A third guide passage or third guide 1305 in which the belt 1250 and the wire 1350 are accommodated and moved may be formed within the second support 1300. The wire winder 1330 may be installed within an end of the second support 1300 at an end of the third guide 1305. The wire winder 1330 may wind or unwind the wire 1350, and may be connected to the second end of the belt 1250. Further, an insertion hole or buckle opening 1332 may be formed at the end of the second support 1300 to couple the buckle 1230 to the wire winder 1330 (FIGS. 13-14). Detailed descriptions of the buckle 1230 and the wire winder 1330 will be described later with reference to FIG. 12.

The first, second, and third guides 1105, 1205, and 1305 may constitute a guide passage. There may be corresponding slots or openings in the main, first, and second supports 1100, 1200, and 1300 to connect the first, second, and third guides 1105, 1205, and 1305 so that the belt 1250 and/or wire 1350 may freely pass through the first, second, and third guides 1105, 1205, and 1305.

The first hinge brackets 1210 and 1210′ may be installed at the first end of the main waist support 1100 to enable hinge coupling of the first support 1200. The first hinge brackets 1210 and 1210′ may be configured in pair, and may be installed on upper and lower sides of the first end of the central support 1110, respectively, and the first support 1200 may be hingedly coupled thereto. The first support 1200 may include a hinge shaft or pin and/or a hinge knuckle that is inserted between the first hinge brackets 1210 and 1210′, and a coupling of the second support 1300 may be substantially similar. Details of the hinge structures may be found in Attorney Docket Ser. No. 16/352,920 (DAE-0086) filed on Mar. 14, 2019, the entire contents of which is incorporated by reference herein.

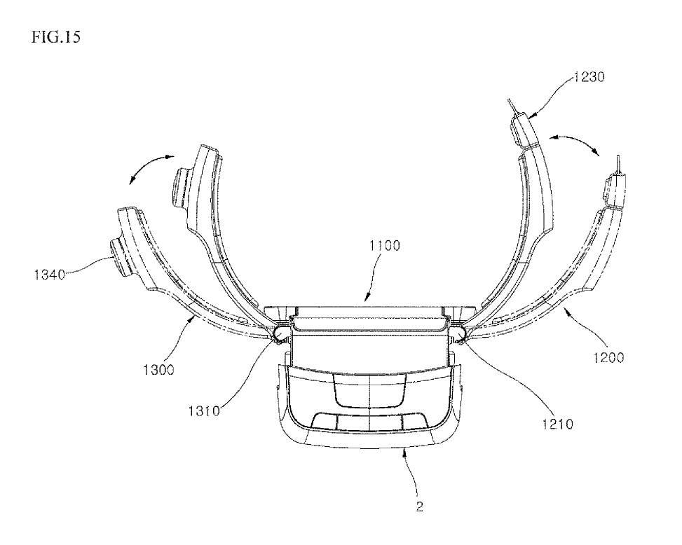

Referring to FIG. 15, the first support 1200 may be pivotable in forward and backward directions within a predetermined angle range by the first hinge brackets 1210 and 1210′. The first hinge brackets 1210 and 1210′ may be provided with a first stopper 1211 that may protrude backward so as to restrict a backward pivotable range of motion of the first support 1200. The first support 1200 may be caught by an edge of the first end of the central support 1110 when it pivots forward, and as a result, a forward pivotable range of the first support 1200 may also may be restricted. When the user secures his waist to the subframe 5 to wear the exoskeleton A, the first support 1200 may naturally tilt forward and backward within the predetermined angle range in accordance with the waist of the user.

Accordingly, the first support 1200 may adapt to and accommodate various waist sizes and shapes. The second hinge brackets 1310 and 1310′ may be installed at the second end of the main waist support 1100 to enable hinge coupling of the second support 1300. The second hinge brackets 1310 and 1310′ may be configured in pair, and may be installed on upper and lower sides of the second end of the central support 1110, respectively, and the second support 1200 may be hingedly coupled thereto. A coupling of the seconds support 1300 to the second hinge brackets 1310 and 1310′ may be substantially the same as a coupling of the first support 1200 to the first hinge brackets 1210 and 1210′.

The second support 1300 may be pivotable in forward and backward directions within a predetermined angle range by the second hinge brackets 1310 and 1310′ and the central support 1110. The second hinge brackets 1310 and 1310′ may be provided with a second stopper 1311 that may protrude rearward so as to restrict a backward pivotable range of the second support 1300. In addition, the second support 1300 may be caught by an edge of the second end of the central support 1110 when pivoting forward, and as a result, a forward pivotable range also may be restricted. When the user secures his waist to the subframe 5 to wear the exoskeleton A, the second support 1300 may naturally tilt forward and backward within the predetermined angle range in accordance with the waist of the user. Accordingly, the second support 1300 may be flexible and adapt to or accommodate various waist sizes and shapes.

Referring back to FIG. 12, the buckle 1230 may be detachably installed at the end of the first support 1200, and the first end of the belt 1250 may be coupled thereto. The buckle 1230 may include a buckle body or base 1233 that has a first end coupled to the belt 1250 and a second end coupled to a buckle plate or a buckle plate or frame 1236. The buckle plate 1236 may be formed with a latch ring or slit 1237. The buckle plate 1326 and latch ring 1327 configuration may also be referred to as a buckle tongue.

The belt 1250 may be accommodated in the first, second, and third guides 1105, 1205, and 1305, and may be made of a material having a tensile or elastic force. When the belt 1250 is withdrawn out of the first support 1200 through the belt hole 1232, the belt 1250 may partially remain in the second guide 1205.

Further, a portion of the buckle plate 1236 may be coupled to the second end of the buckle base 1233. The latch ring 1237 may be provided in a portion of the buckle plate 1236 that protrudes outward from the buckle base 1233.

The wire winder 1330 may be installed within the end of the second support 1300 to wind or unwind the wire 1350 connected to the second end of the belt 1250. The wire winder 1330 may include a winding body or a case 1338 provided with a spring or elastic member 1339 (e.g., leaf spring or plate spring) around which the wire 1350 is wound, a buckle receiver or latch assembly 1336 provided at an end of the case 1338 and positioned near the end of the second support 1300, and a button dial or knob 1340 that may be provided on an outer surface of the case 1338 to protrude from the second support 1300. The case 1338 may include a wire winding dispenser, which may be one of a bobbin, reel, and spindle (not shown). The latch assembly 1336 may be provided with a latch hook 1337. The button dial 1340 may be withdrawable and insertable. The spring 1339 may be a plate spring similar to the plate spring disclosed in U.S. application Ser. No. 16/352,920 (Attorney Docket No. DAE-0086) filed on Mar. 14, 2019, the entire contents of which is incorporated by reference herein. An alternative embodiment may not include a plate spring, and may instead include a leaf spring.

The wire 1350 may be accommodated primarily in the first and third guides 1105 and 1305, and may be made of a metal material or a reel. When the belt 1250 is withdrawn out of the first support 1200 through the belt hole 1232, the wire 1350 connected to the belt 1250 may be unwound from the wire winder 1330. As a result, the wire 1350 may be provided in the first to third guides 1105, 1205, and 1305.

The spring 1339 provided in the case 1338 may have a circular, semicircular, or curved shape, and may pull the wire 1350. The case 1338 may be connected to or separated from the button dial 1340 in response to withdrawal or insertion of the button dial 1340. When the case 1338 is connected to the button dial 1340, the case 1338 (or a reel within the case 1338) may rotate with a rotation of the button dial 1340.

For example, when the user or the assistant rotates the button dial 1340 in a first direction, such as clockwise, the case 1338 or a reel therein coupled to the button dial 1340 may also rotate in the first direction. The spring 1339 provided in the case 1338 may be contracted or compressed, and as a result, the wire 1350 may be wound.

When the user or the assistant rotates the button dial 1340 in a second direction opposite to the first direction, such as counterclockwise, the case 1338 coupled to the button dial 1340 or the reel within the case 1338 may also rotate in the second direction. The spring 1339 provided in the case 1338 may be expanded or released, and as a result, the wire 1350 may be unwound.

Referring to FIG. 13, when the user or the assistant inserts the button dial 1340 inward in a Lock “L” direction, or withdraws the button dial 1340 outward in an Unlock “UL” direction, the latch hook 1337 may be fastened to or detached from the latch ring 1237, respectively. The button dial 1340 may be withdrawable upward or outward, or withdrawable in a “UL” direction by various increments or steps. The user may withdraw the button dial 1340 by a first step or increment to a first position, and then a second step or increment beyond the first increment to a second position, etc. A length of the first increment may be equal to a length of the second increment. The second position may be further outward than the first position.

As shown in FIGS. 13 and 14, when the buckle 1230 is pulled off of or detached from the end of the first support 1200 and inserted into the insertion hole 1332 to secure the waist of the user, the belt 1250 may be withdrawn out of the first support 1200 through the belt hole 1232, and the buckle 1230 may be coupled to the wire winder 1330 installed within the end of the second support 1300. The buckle plate 1236 may be coupled to the latch assembly 1336, and the latch ring 1237 of the buckle plate 1236 may be fastened to the latch hook 1337 of the latch assembly 1336.

When the user withdraws the button dial 1340 outward in a “UL” direction by a first step, the button dial 1340 may detach from the case 1338, and accordingly, the spring 1339 provided in the case 1338 may release the wire 1350. As a result, the wire 1350 wound around the spring 1339 may be unwound. Thus, the user may pull the buckle 1230. Also, when the pulled buckle 1230 is inserted into the insertion hole 1332, the latch assembly 1336 may be tilted by the buckle plate 1236 so that the latch hook 1337 may be fastened to the latch ring 1237. As a result, the user may easily secure the exoskeleton A at his waist. Details of the buckle 1230, the latch assembly 1336, and other details of the waist belt are disclosed in U.S. application Ser. No. 16/352,920 (Attorney Docket No. DAE-0086) filed on Mar. 14, 2019, the entire disclosure of which is incorporated by reference herein.

When the button dial 1340 is inserted inward in an “L” direction when the buckle plate 1236 is coupled to the latch assembly 1336, the button dial 1340 and the case 1338 may couple to each other. When the button dial 1340 is rotated in the winding direction of the wire 1350 when the button dial 1340 and the case 1338 are connected to each other, the wire 1350 may be wound around the spring 1339 within a tensile force range of the belt 1250 so that the belt 1250 may tighten around the waist of the user. As a result, the user may adjust a tightening of the belt 1250.

On the other hand, when the button dial 1340 is withdrawn outward in a “UL” direction again when the buckle plate 1236 is coupled to the latch assembly 1336, the latch hook 1337 of the latch assembly 1336 may be detached from the latch ring 1237 of the buckle plate 1236, so that the wire 1350 may be rewound around the spring 1339 to retract the belt 1250.

The user may withdraw the button dial 1340 in an Unlock “UL” direction by a second step, and a shaft that fixes the latch assembly 1336 may be interlocked with the button dial 1340 and pulled in a “UL” direction so that the latch hook 1337 is detached from the latch ring 1237. As a result, the buckle 1230 may also be detached from the insertion hole 1332, and the unwound wire 1350 may be automatically rewound around the spring 1339 by a restoring force of the spring 1339 provided in the case 1338. As a result, the user may easily undo the belt 1250. Also, since the belt 1250 may be automatically wound and accommodated within the subframe 5, the user or the assistant may prepare for the next session or donning without any additional work or preparation.

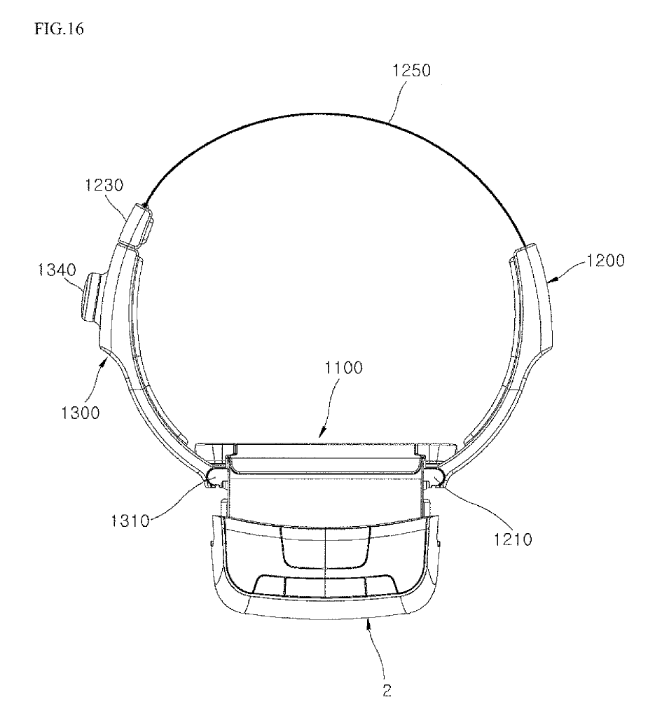

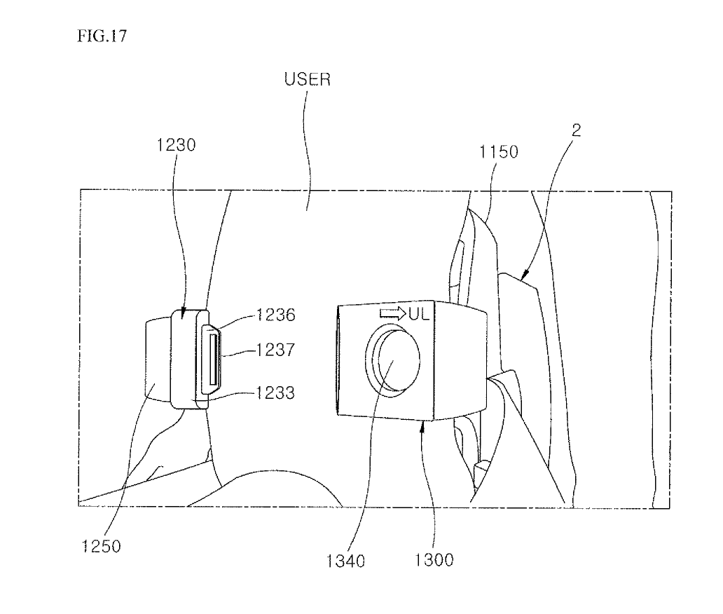

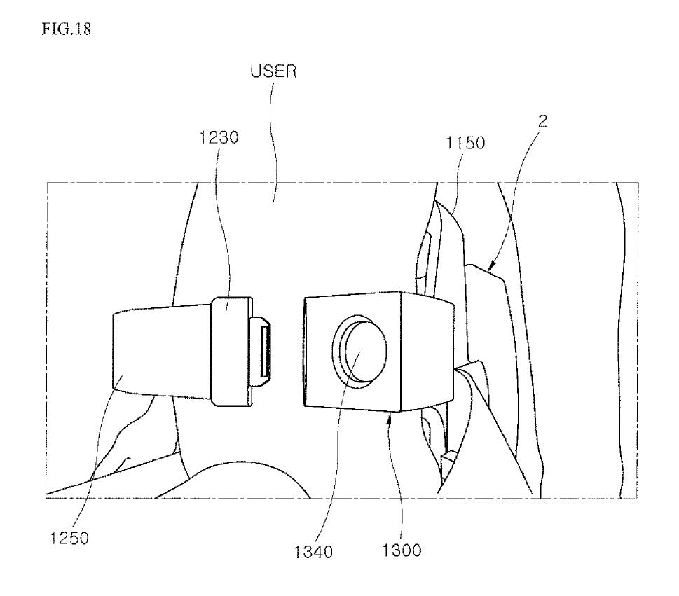

Hereinafter, a process of securing the subframe 5 of the above-described exoskeleton A to the waist of the user will be described in more detail with reference to FIG. 15 to FIG. 19. FIGS. 15 and 16 are schematic views explaining a method of securing a waist of a user to the subframe of FIG. 7. FIGS. 17 to 19 are schematic views explaining a method of using a dial according to putting on or taking off a belt.

As shown in FIG. 15, the first and second supports 1200 and 1300 may be pivotable forward and backward within a predetermined angle range by first and second hinge brackets 1210, 1210′, 1310, and 1310′, as previously described. The user may fit the first and second supports 1200 and 1300 onto his waist in accordance with his waist size and shape.

Referring to FIG. 16, when a fitting process is completed, the user may detach or pull the buckle 1230 from the end of the first support 1200 and insert the detached buckle 1230 into the insertion hole 1332 (FIG. 12) of the second support 1300. The buckle plate 1236 may be coupled to the latch assembly 1336 through the insertion hole 1332 so that the waist of the user may be secured by the subframe 5.

Hereinafter, a method of using the dial according to a buckling and unbuckling (or putting on and taking off) the belt 1250 will be described with reference to FIG. 12 and FIGS. 17 to 19. When the fitting process shown in FIG. 15 is completed, the button dial 1340 may be withdrawn in a “UL” direction by a first increment to a first position, and the user (USER) may detach the buckle 1230 from the first support 1200, thereby withdrawing the belt 1250 from the subframe 5.

The user (USER) may also adjust the withdrawn belt 1250 to extend across his entire waist, and then insert the buckle plate 1236 of the buckle 1230 into the insertion hole 1332 of the second support 1300, thereby fastening the latch hook 1337 to the latch ring 1237.

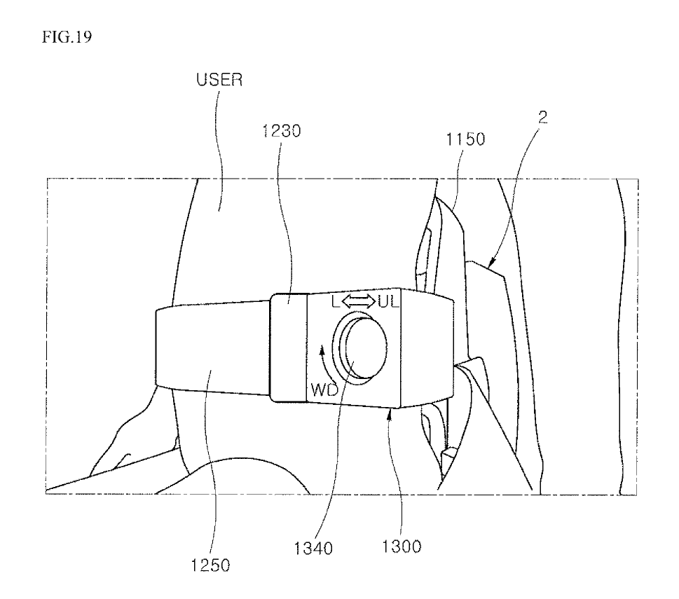

Referring to FIG. 19, the user (USER) may insert the button dial 1340 inward by the L direction and rotate the button dial 1340 in a winding direction “WD” of the wire 1350, thereby tightening the belt 1250 more firmly in accordance with his waist size. The user (USER) may also rotate the button type dial 1340 in an unwinding direction of the wire 1350 opposite to the “WD” direction to loosen the belt 1250.

When the user (USER) finishes using the exoskeleton A, the user (USER) may detach the latch hook 1337 from the latch ring 1237 by withdrawing the button dial 1340 outward in the “UL” direction by a second increment (or by two increments) to a second position. The second position may be the further outward than the first position. When the latch hook 1337 is detached from the latch ring 1237, the wire 1350 connected to the belt 1250 may be automatically wound around the spring 1339 provided in the case 1338 so that the belt 1250 may automatically retract back into the subframe 5.

Buckling and unbuckling the belt 1250 may be a three step process. In the first step, the user may pull the button dial 1340 outward and withdraw the belt 1250 from the first support 1200. In the second step, the user may insert the buckle 1230 into the second support 1300, and the buckle 1230 may fasten via a coupling of the latch ring 1237 and the latch hook 1337. Once the buckle 1230 is fastened, the user may push the button dial 1340 inward and rotate the button dial 1340 to adjust a tightness of the belt 1250. In the third step, the user may pull the button dial 1340 outward again, which will release the buckle 1230 and retract the belt 1250 back into the first support 1200.

As described above, a wearable assistive device such as a wearable robot A, e.g., exoskeleton, according to an embodiment may secure to a waist of a user and thus may provide stability. Accordingly, the user may stably walk, bend, or lift with the aid of an assistive force.

In the wearable assistive device according to an embodiment, a waist supporting structure or a waist support may easily secure to or detach from the waist of the user, and thus the user may easily wear or take off the wearable assistive device. Also, since the time required for donning or removing the wearable assistive device is reduced, more patients may use the wearable assistive device in a set amount of time, thereby improving the profitability of a hospital in which the wearable assistive device is used.

In addition, the wearable assistive device according to an exemplary embodiment may adapt to or accommodate various waist sizes and shapes, and thus may be universally used regardless of gender or age, and convenience may also be improved. Furthermore, the wearable assistive device may be designed as a general-purpose type that accommodates a wide variety of users, instead of being personalized and/or limited to a specific user, thereby improving the profitability of the hospital or facility in which the wearable assistive device is used. Since various substitutions, changes, and modifications can be made within the scope that does not deviate the technical idea of this application for those skilled in the art to which this application pertains, embodiments disclosed herein are not limited by the above-mentioned embodiments and the accompanying drawings.

Embodiments disclosed herein may provide a wearable assistive device such as a wearable robot, e.g., exoskeleton, capable of stably securing a waist of a user while minimizing instability. The wearable assistive device may be capable of easily securing or detaching a waist supporting structure or waist support. The wrist support may be part of a structure a subframe unit or subframe.

Embodiments disclosed herein may provide a wearable assistive device that may flexibly deal with various waist sizes and shapes. Embodiments disclosed herein are not limited to the above-mentioned objects, and other objects and advantages which are not mentioned can be understood by the description, and more clearly understood by the embodiments disclosed herein. It will be also be readily seen that the objects and the advantages disclosed herein may be realized by means indicated in the patent claims and a combination thereof.

The wearable assistive device may secure the waist of the user while minimizing shaking or instability, and thus may provide the user with a stable assistive force. Therefore, the user may stably walk, bend, or lift with the aid of the assistive force. The wearable assistive device may include a waist support, which may easily be secured to or detached from the waist of the user, and thus the user may easily don or take off the wearable assistive device. Since a time required to wear or take off the wearable assistive device may be minimized, more people (e.g., patients) may use the wearable assistive device in a certain amount of time, thereby improving the profitability of a facility (e.g. hospital) in which the wearable assistive device is used.

In addition, the wearable assistive device may adapt to or accommodate various waist sizes and shapes, and thus may be universally used regardless of gender or age, improving convenience. Furthermore, the wearable assistive device may be designed more generally and does not need to be personalized, thereby improving the profitability of the facility in which the wearable assistive device is used. Further details on the waist support may be found in U.S. application Ser. No. 16/274,697 (Attorney Docket No. DAE-0077) filed on Feb. 13, 2019, the entire contents of which is incorporated by reference herein.

It will be understood that when an element or layer is referred to as being “on” another element or layer, the element or layer can be directly on another element or layer or intervening elements or layers. In contrast, when an element is referred to as being “directly on” another element or layer, there are no intervening elements or layers present. As used herein, the term “and/or” includes any and all combinations of one or more of the associated listed items.

It will be understood that, although the terms first, second, third, etc., may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section could be termed a second element, component, region, layer or section without departing from the teachings of the present disclosure.

Spatially relative terms, such as “lower”, “upper” and the like, may be used herein for ease of description to describe the relationship of one element or feature to another element(s) or feature(s) as illustrated in the figures. It will be understood that the spatially relative terms are intended to encompass different orientations of the device in use or operation, in addition to the orientation depicted in the figures. For example, if the device in the figures is turned over, elements described as “lower” relative to other elements or features would then be oriented “upper” relative the other elements or features. Thus, the exemplary term “lower” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the disclosure are described herein with reference to cross-section illustrations that are schematic illustrations of idealized embodiments (and intermediate structures) of the disclosure. As such, variations from the shapes of the illustrations as a result, for example, of manufacturing techniques and/or tolerances, are to be expected. Thus, embodiments of the disclosure should not be construed as limited to the particular shapes of regions illustrated herein but are to include deviations in shapes that result, for example, from manufacturing.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Any reference in this specification to “one embodiment,” “an embodiment,” “example embodiment,” etc., means that a particular feature, structure, or characteristic described in connection with the embodiment is included in at least one embodiment of the disclosure. The appearances of such phrases in various places in the specification are not necessarily all referring to the same embodiment. Further, when a particular feature, structure, or characteristic is described in connection with any embodiment, it is submitted that it is within the purview of one skilled in the art to effect such feature, structure, or characteristic in connection with other ones of the embodiments.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. A wearable assistive device, comprising:
a main frame having two ends and formed to cover a pelvis;
a subframe coupled to the main frame for securing to a waist;
a leg assembly installed at each end of the main frame and formed to extend downward along a leg; wherein the subframe comprises:
a main waist support that supports a lumbar;
at least one first arm that is hingedly coupled to a first end of the main waist support to wrap over a first side of the waist; and
at least one second arm that is hingedly coupled to a second end of the main waist support to warp over a second side of the waist.

2. The wearable assistive device of claim 1, wherein the subframe further comprises:
a first hinge bracket installed at the first end of the main waist support to enable hinge coupling of the at least one first arm; and
a second hinge bracket installed at the second end of the main waist support to enable hinge coupling of the at least one second arm.

3. The wearable assistive device of claim 2, wherein the at least one first arm is pivotable within a first predetermined angle range at the first hinge bracket, and
wherein the at least one second arm is pivotable within a second predetermined angle range at the second hinge bracket.

4. The wearable assistive device of claim 1, further including:
a first impact cushion made of a soft foam material attached to an inner surface of the at least one first arm, and
a second impact cushion made of a soft foam material attached to an inner surface of the at least one second arm.

5. The wearable assistive device of claim 4, wherein the first and second impact cushions are attached to the inner surfaces of the first and second arms in a Velcro manner.

6. The wearable assistive device of claim 1, wherein the subframe further comprises:
a belt buckle that is detachably installed at an end of the at least one first arm, wherein a first end of a belt is coupled to the belt buckle; and
a wire winder installed in an end of the at least one second arm to wind or unwind a wire connected to a second end of the belt, wherein the belt and the wire are accommodated in a guide passage that is formed within the main waist support and the first and second arms.

7. The wearable assistive device of claim 6, wherein the guide passage comprises:
a first guide formed within the main waist support;
a second guide formed within the at least one first arm; and
a third guide formed within the at least one second arm.

8. The wearable assistive device of claim 6, wherein the belt is made of a material having a tensile force, and the wire is made of a metal material.

9. The wearable assistive device of claim 6,
wherein a belt opening from which the belt is withdrawn is formed at the end of the at least one first arm, and
wherein a buckle opening through which the belt buckle is inserted is formed at the end of the at least one second arm.

10. The wearable assistive device of claim 9, wherein, when the belt buckle is detached from the end of the at least one first arm and inserted into the buckle opening to secure the waist of the user, the belt is withdrawn out of the first arm through the belt opening, and the belt buckle is coupled to the wire winder.

11. The wearable assistive device of claim 6, wherein the wire winder comprises a case provided with a spring around which the wire is wound, a latch assembly provided at an end of the case near the end of the at least one second arm and including a latch hook, and a button dial provided on an outer surface of the case to protrude from the second arm, wherein the button dial is withdrawable and insertable; and wherein the belt buckle comprises a buckle base that has a first end coupled to the belt, and a buckle plate that is coupled to a second end of the buckle base to protrude, the buckle plate including a latch ring.

12. The wearable assistive device of claim 11, wherein the buckle plate is coupled to the latch assembly through the buckle opening, and the latch ring of the buckle plate is fastened to the latch hook of the latch assembly when the belt buckle is detached from the end of the at least one first arm and inserted into the buckle opening at the end of the at least one second arm to secure the belt across the waist of the user.

13. The wearable assistive device of claim 12, wherein, when the buckle plate is coupled to the latch assembly and the button dial is withdrawn, the latch hook is detached from the latch ring and the wire is rewound around the spring.

14. The wearable assistive device of claim 12, wherein, when the buckle plate is coupled to the latch assembly, the button dial is inserted, and when the button dial is rotated in a winding direction of the wire, the wire is wound around the spring within a tensile force range of the belt.

15. The wearable assistive device of claim 1, wherein the subframe further comprises a handle that is installed at an upper end of the main waist support and is bent in a parabolic shape.

16. A wearable assistive device, including:
a main frame coupled to a subframe, the subframe provided at a waist or trunk; and
a leg assembly that extends from the main frame and is coupled to a leg, wherein the subframe includes:
a central support,
a first side assembly that curves around a first side of a user and is rotatably coupled to a first side of the central support, and,
a second side assembly that curves around a second side of a user and is rotatably coupled to a second side of the central support.

17. The wearable assistive device of claim 16, wherein an interior of the subframe stores a belt coupled to a wire, the wire is wound and unwound via a wire winder installed in the second side assembly, the belt slides in and out of a first slot installed in the first side assembly, and a length of a withdrawn portion of the belt that is outside the frame is adjustable via a winding of the wire around the wire winder.

18. The wearable assistive device of claim 17, wherein the belt is made of a tensile or stretchy material, the wire is made of a metal material, and wherein the belt includes a tongue made of a rigid material that is inserted into and fixed inside of a second slot of the second side assembly so that the belt detachably couples to the second side assembly.

19. The wearable assistive device of claim 16, wherein the central support includes a first stopper that restricts a rotation of the first side assembly around a first hinge that couples the first side assembly to the central support, and a second stopper that restricts a rotation of the second side assembly around a second hinge that couples the second side assembly to the central support.

20. The wearable assistive device of claim 16, further including a retractable belt that couples the first side assembly to the second side assembly such that a size or tightness of the subframe can be adjusted via a rotation of the first and second side assemblies and the retractable belt.
